(19) United States
(12) Patent Application Publication
(10) Pub. No.: US 2019/0071720 A1
(43) Pub. Date: Mar. 7, 2019

(54) DEVICES, SYSTEMS AND METHODS FOR NUCLEIC ACID SEQUENCING

(71) Applicant: Quantum Biosystems Inc., Tokyo (JP)

(72) Inventors: Eric S. Nordman, Palo Alto, CA (US); Mark Oldham, Emerald Hills, CA (US); Takeshi Akatsu, Tokyo (JP)

(21) Appl. No.: 15/937,327

(22) Filed: Mar. 27, 2018

Related U.S. Application Data
(63) Continuation of application No. PCT/JP2016/004531, filed on Oct. 11, 2016.
(60) Provisional application No. 62/239,171, filed on Oct. 8, 2015.

(51) Int. Cl. C12Q 1/6869

(52) U.S. Cl. CPC C12Q 1/6869; C12Q 2563/157; C12Q 2563/159; C12Q 2565/631; C12Q 2563/116; C12Q 2565/507

(57) ABSTRACT

The present disclosure provides devices, systems and methods for sequencing nucleic acid molecules. A sequence of a nucleic acid molecule may be identified at high accuracy using a chip comprising an array of sensors, wherein each individual sensor comprises at least one nano-gap electrode pair configured to generate electrical signals upon flow of the nucleic acid molecule through or in proximity to at least one nano-gap of the nano-gap electrode pair.

### CROSS-REFERENCE

This application is a continuation of International Patent Application PCT/JP2016/004531, filed Oct. 11, 2016, which claims the benefit of U.S. Provisional Patent Application No. 62/239,171, filed Oct. 8, 2015, each of which is entirely incorporated herein by reference.

### BACKGROUND

Nucleic acid sequencing is the process of determining the order of nucleotides within a nucleic acid molecule, such as deoxyribonucleic acid (DNA) or ribonucleic acid (RNA). The determination of the sequence of a nucleic acid molecule may provide various benefits, such as aiding in diagnosing and/or treating a subject. For example, the nucleic acid sequence of a subject may be used to identify, diagnose and potentially develop treatments for genetic diseases.

### SUMMARY

While there are nucleic acid sequencing methods and systems presently available, recognized herein are various limitations associated with such systems, for example, they may be expensive or may not provide sufficient sequence information within a time period and at an accuracy that may be necessary to diagnose and/or treat a subject.

The present disclosure provides methods, systems and computer programs that may be useful for species identification and/or nucleic acid sequencing. Such methods and systems may be capable of performing high-accuracy sequencing by sequencing nucleic acid molecules (e.g., DNA, RNA or variant thereof) independently or in parallel, using highly sensitive signals, which signals may be standardized.

An aspect of the present disclosure provides a method for sequencing a nucleic acid molecule, comprising: (a) providing a chip comprising an array of individual sensors, wherein each individual sensor of the array comprises a solid state membrane having at least one nano-gap, wherein the at least one nano-gap comprises electrodes that are adapted to generate an electrical signal to aid in detection of the nucleic acid molecule or a portion thereof upon flow of the nucleic acid molecule or a portion thereof through the at least one nano-gap; (b) directing the nucleic acid molecule or a portion thereof through or in proximity to the at least one nano-gap; and (c) identifying a nucleic acid sequence of the nucleic acid molecule or a portion thereof at an accuracy of at least about 97%.

In some embodiments of aspects provided herein, the nucleic acid sequence of the nucleic acid molecule or portion thereof is determined at an accuracy of at least about 97% over a span of at least about 100 contiguous nucleic acid bases of the nucleic acid molecule. In some embodiments of aspects provided herein, the nucleic acid sequence of the nucleic acid molecule or portion thereof is determined at an accuracy of at least about 97% in the absence of re-sequencing the nucleic acid molecule or a portion thereof. In some embodiments of aspects provided herein, the electrodes are coupled to an electric circuit. In some embodiments of aspects provided herein, the sensors are coupled to an integrated circuit that processes the electrical signal. In some embodiments of aspects provided herein, the sensors are part of the chip. In some embodiments of aspects provided herein, the array of sensors comprises individual sensors at a density greater than or equal to about 50 or 500 individual sensors per 1 mm². In some embodiments of aspects provided herein, each of the individual sensors is independently addressable. In some embodiments of aspects provided herein, the nucleic acid molecule comprises deoxyribonucleic acid (DNA) and/or ribonucleic acid RNA. In some embodiments of aspects provided herein, the solid state membrane is at least partially formed of at least one material selected from the group consisting of a metallic material and a semiconductor material. In some embodiments of aspects provided herein, the solid state membrane is at least partially formed of a material selected from the group consisting of silicon nitrides, silica and alumina. In some embodiments of aspects provided herein, the solid state membrane has a thickness in the range of about 10 nanometers (nm) to about 1 millimeter (mm). In some embodiments of aspects provided herein, the solid state membrane has an inter-electrode capacitance of less than about 0.1 picofarad (pF). In some embodiments of aspects provided herein, the accuracy is at least about 99.5%. In some embodiments of aspects provided herein, the accuracy is at least about 97% when identifying up to 5 nucleic acid bases of the nucleic acid molecule or a portion thereof. In some embodiments of aspects provided herein, the accuracy is at least about 97% when identifying up to 3 nucleic acid bases of the nucleic acid molecule or a portion thereof. In some embodiments of aspects provided herein, the accuracy is at least about 97% when identifying a single nucleic acid base of the nucleic acid molecule or a portion thereof. In some embodiments of aspects provided herein, the accuracy of at least about 97% is achieved by combining data collected from at most 20 passes of the nucleic acid molecule or a portion thereof through the at least one nano-gap. In some embodiments of aspects provided herein, the accuracy of at least about 97% is achieved by combining data collected from at most 5 passes of the nucleic acid molecule or a portion thereof through the at least one nano-gap. In some embodiments of aspects provided herein, the accuracy of at least about 97% is achieved by combining data collected from a single pass of the nucleic acid molecule or a portion thereof through the at least one nano-gap. In some embodiments of aspects provided herein, the nucleic acid sequence of the nucleic acid molecule or a portion thereof is identified by combining data collected from at least 10 passes of the nucleic acid molecule or a portion thereof through the at least one nano-gap. In some embodiments of aspects provided herein, the nucleic acid sequence of the nucleic acid molecule or a portion thereof is identified by combining data collected from at least 20 passes of the nucleic acid molecule or a portion thereof through the at least one nano-gap. In some embodiments of aspects provided herein, the electrical signal comprises current. In some embodiments of aspects provided herein, the nucleic acid molecule is directed through the at least one nano-gap at a translocation rate of at least about 0.5 kilohertz (KHz) at a current level of at least about 1 picoampere, a background current level of at least about 1 nanoampere, and a signal to noise ratio of at least about 1 to 2. In some embodiments of aspects provided herein, the electrodes comprise tunneling electrodes. In some embodiments of aspects provided herein, the electrical signal comprises tunneling current. In some embodiments of aspects provided herein, the nucleic acid sequence of the nucleic acid molecule or a portion thereof is identified at a frequency from about 0.1 kilohertz (KHz) to 100 KHz. In some embodiments of aspects provided herein, the electrodes are spaced apart by a gap having a spacing from about 0.5 times to five times a molecular diameter of a given nucleic acid subunit of the nucleic acid molecule. In some embodiments of aspects provided herein, the electrodes are spaced apart by a gap having a spacing from about 0.5 times to two times a molecular diameter of a given nucleic acid subunit of the nucleic acid molecule. In some embodiments of aspects provided herein, the electrodes are spaced apart by a gap having a spacing from about 0.5 times to less than or equal to a molecular diameter of a given nucleic acid subunit of the nucleic acid molecule. In some embodiments of aspects provided herein, the identifying comprises generating a raw accuracy of at least about 80%. In some embodiments of aspects provided herein, the identifying comprises generating a consensus sequence. In some embodiments of aspects provided herein, the nucleic acid sequence of the nucleic acid molecule or a portion thereof is identified at a single pass accuracy of at least about 80%.

Another aspect of the present disclosure provides a method for sequencing a nucleic acid molecule, comprising: (a) providing a chip comprising an array of individual sensors, wherein each individual sensor of the array comprises a solid state membrane having at least one nano-gap, wherein the at least one nano-gap comprises electrodes that are adapted to generate an electrical signal to aid in detection of the nucleic acid molecule or a portion thereof upon flow of the nucleic acid molecule or a portion thereof through the at least one nano-gap; (b) directing flow of the nucleic acid molecule or a portion thereof through or in proximity to the at least one nano-gap in the absence of a molecular motor; and (c) sequencing the nucleic acid molecule by detecting the electrical signal at multiple time points.

In some embodiments of aspects provided herein, the electrodes are coupled to an electric circuit. In some embodiments of aspects provided herein, the sensors are coupled to an integrated circuit that processes the electrical signal. In some embodiments of aspects provided herein, the sensors are part of the chip. In some embodiments of aspects provided herein, the array of sensors comprises individual sensors at a density greater than or equal to about 500 individual sensors per 1 mm². In some embodiments of aspects provided herein, each of the individual sensors is independently addressable. In some embodiments of aspects provided herein, the nucleic acid molecule comprises deoxyribonucleic acid (DNA) and/or ribonucleic acid (RNA). In some embodiments of aspects provided herein, the solid state membrane is formed of at least one material selected from the group consisting of a metallic material and a semiconductor material. In some embodiments of aspects provided herein, the solid state membrane is formed of a material selected from the group consisting of silicon nitrides, silica and alumina. In some embodiments of aspects provided herein, the solid state membrane has a thickness in the range of about 10 nanometers (nm) to about 1 milliliter (mm). In some embodiments of aspects provided herein, the solid state membrane has a capacitance of less than about 0.1 picofarad (pF). In some embodiments of aspects provided herein, the nucleic acid molecule is sequenced at an accuracy of at least about 95%. In some embodiments of aspects provided herein, the electrical signal comprises current. In some embodiments of aspects provided herein, the electrodes comprise tunneling electrodes. In some embodiments of aspects provided herein, the electrical signal comprises tunneling current. In some embodiments of aspects provided herein, the nucleic acid molecule is sequenced by detecting one or more nucleic acid subunits of the nucleic acid molecule upon flow of the nucleic acid molecule or a portion thereof through the at least one nano-gap. In some embodiments of aspects provided herein, the one or more nucleic acid subunits are detected at a signal to noise ratio of at least about 10-to-1, 50-to-1, or 100-to-1. In some embodiments of aspects provided herein, individual subunits of the nucleic acid molecule are detected in a time period of at most about 1 microsecond or 1 millisecond. In some embodiments of aspects provided herein, the electrodes are spaced apart by a gap having a spacing from about 0.5 times to five times a molecular diameter of a given nucleic acid subunit of the nucleic acid molecule. In some embodiments of aspects provided herein, the electrodes are spaced apart by a gap having a spacing from about 0.5 times to two times a molecular diameter of a given nucleic acid subunit of the nucleic acid molecule. In some embodiments of aspects provided herein, the electrodes are spaced apart by a gap having a spacing from about 0.5 times to less than or equal to a molecular diameter of a given nucleic acid subunit of the nucleic acid molecule.

Another aspect of the present disclosure provides a system for sequencing a nucleic acid molecule, comprising: (a) a chip comprising an array of individual sensors, wherein each individual sensor of the array comprises a solid state membrane configured to have at least one nano-gap therein, wherein the at least one nano-gap comprises electrodes coupled to an electrical circuit that is adapted to generate an electrical signal(s) to aid in detection of the nucleic acid molecule or a portion thereof upon flow of the nucleic acid molecule or a portion thereof through the at least one nano-gap; and (b) a computer processor coupled to the chip, wherein the computer processor is programmed to aid in characterizing a nucleic acid sequence of the nucleic acid molecule or the portion thereof based on the electrical signal(s) received from the array of individual sensors at an accuracy of at least about 97%.

In some embodiments of aspects provided herein, the nucleic acid sequence of the nucleic acid molecule or portion thereof is determined at an accuracy of at least about 97% over a span of at least about 100 contiguous nucleic acid bases of the nucleic acid molecule. In some embodiments of aspects provided herein, the nucleic acid sequence of the nucleic acid molecule or portion thereof is determined at an accuracy of at least about 97% in the absence of re-sequencing the nucleic acid molecule or a portion thereof. In some embodiments of aspects provided herein, the array of individual sensors is at a density of at least about 500 individual sensors per 1 mm². In some embodiments of aspects provided herein, each of the individual sensors is independently addressable. In some embodiments of aspects provided herein, the nucleic acid molecule comprises deoxyribonucleic acid (DNA) and/or ribonucleic acid (RNA). In some embodiments of aspects provided herein, the solid state membrane is formed of at least one material selected from the group consisting of a metallic material and a semiconductor material. In some embodiments of aspects provided herein, the solid state membrane is formed of a material selected from the group consisting of silicon nitrides, silica and alumina. In some embodiments of aspects provided herein, the solid state membrane has a thickness in the range of about 10 nanometers (nm) to about 1 millimeter (mm). In some embodiments of aspects provided herein, the solid state membrane has a capacitance of less than about 0.1 picofarad (pF). In some embodiments of aspects provided herein, the electrical signal comprises current. In some embodiments of aspects provided herein, the electrodes comprise tunneling electrodes. In some embodiments of aspects provided herein, the electrical signal comprises tunneling current. In some embodiments of aspects provided herein, the electrodes are spaced apart by a gap having a spacing from about 0.5 times to five times a molecular diameter of a given nucleic acid subunit of the nucleic acid molecule. In some embodiments of aspects provided herein, the electrodes are spaced apart by a gap having a spacing from about 0.5 times to two times a molecular diameter of a given nucleic acid subunit of the nucleic acid molecule. In some embodiments of aspects provided herein, the electrodes are spaced apart by a gap having a spacing from about 0.5 times to less than or equal to a molecular diameter of a given nucleic acid subunit of the nucleic acid molecule. In some embodiments of aspects provided herein, the system further comprises a transimpedence amplifier operatively coupled to the computer processor.

Another aspect of the present disclosure provides a system for sequencing a nucleic acid molecule, comprising: (a) a chip comprising an array of individual sensors, wherein each individual sensor of the array comprises a solid state membrane configured to have at least one nano-gap therein, wherein the at least one nano-gap comprises electrodes that are coupled to an electrical circuit adapted to generate an electrical signal to aid in detection of the nucleic acid molecule or a portion thereof upon flow of the nucleic acid molecule or a portion thereof through the at least one nano-gap, and wherein each of the individual sensors is independently addressable; and (b) a computer processor coupled to the chip, wherein the computer processor is programmed to aid in characterizing a nucleic acid sequence of the nucleic acid molecule or a portion thereof by detecting the electrical signal at multiple time points.

In some embodiments of aspects provided herein, the flow of the nucleic acid molecule or a portion thereof is facilitated without the use of a molecular motor. In some embodiments of aspects provided herein, the array of individual sensors is at a density of at least about 500 individual sensors per 1 mm². In some embodiments of aspects provided herein, the nucleic acid molecule comprises deoxyribonucleic acid (DNA) and/or ribonucleic acid (RNA). In some embodiments of aspects provided herein, the solid state membrane is formed of at least one material selected from the group consisting of a metallic material and a semiconductor material. In some embodiments of aspects provided herein, the solid state membrane is formed of at least one material selected from the group consisting of silicon nitrides, silica and alumina. In some embodiments of aspects provided herein, the solid state membrane has a thickness in the range of about 10 nanometers (nm) to about 1 millimeter (mm). In some embodiments of aspects provided herein, the solid state membrane has a capacitance of less than about 0.1 picofarad (pF). In some embodiments of aspects provided herein, the electrical signal comprises current. In some embodiments of aspects provided herein, the electrodes comprise tunneling electrodes. In some embodiments of aspects provided herein, the electrical signal comprises tunneling current. In some embodiments of aspects provided herein, the nucleic acid molecule is sequenced at an accuracy of at least about 95%. In some embodiments of aspects provided herein, the electrodes are spaced apart by a gap having a spacing from about 0.5 times to five times a molecular diameter of a given nucleic acid subunit of the nucleic acid molecule. In some embodiments of aspects provided herein, the electrodes are spaced apart by a gap having a spacing from about 0.5 times to two times a molecular diameter of a given nucleic acid subunit of the nucleic acid molecule. In some embodiments of aspects provided herein, the electrodes are spaced apart by a gap having a spacing from about 0.5 times to less than or equal to a molecular diameter of a given nucleic acid subunit of the nucleic acid molecule.

Another aspect of the present disclosure provides a method for sequencing a nucleic acid molecule, comprising: (a) activating a chip comprising an array of individual sensors, wherein each individual sensor of the array comprises a solid state membrane having at least one nano-gap, wherein the at least one nano-gap comprises electrodes that are adapted to generate an electrical signal to aid in detection of the nucleic acid molecule or a portion thereof upon flow of the nucleic acid molecule or a portion thereof through the at least one nano-gap, wherein the electrodes are spaced apart by a gap having a spacing from about 0.5 times to five times a molecular diameter of a given nucleic acid subunit of the nucleic acid molecule; (b) directing the nucleic acid molecule or a portion thereof through or in proximity to the at least one nano-gap; and (c) identifying a nucleic acid sequence of the nucleic acid molecule or a portion thereof.

In some embodiments of aspects provided herein, the nucleic acid sequence of the nucleic acid molecule or portion thereof is determined at an accuracy of at least about 97% over a span of at least about 100 contiguous nucleic acid bases of the nucleic acid molecule. In some embodiments of aspects provided herein, the nucleic acid sequence of the nucleic acid molecule or a portion thereof is determined at an accuracy of at least about 97% in the absence of re-sequencing the nucleic acid molecule or a portion thereof. In some embodiments of aspects provided herein, the electrodes are coupled to an electric circuit. In some embodiments of aspects provided herein, the sensors are coupled to an integrated circuit that processes the electrical signal. In some embodiments of aspects provided herein, the sensors are part of the chip. In some embodiments of aspects provided herein, the array of sensors comprises individual sensors at a density greater than or equal to about 50 or 500 individual sensors per 1 mm². In some embodiments of aspects provided herein, the nucleic acid molecule is directed through the at least one nano-gap at a translocation rate of at least about 0.5 kilohertz (KHz). In some embodiments of aspects provided herein, the nucleic acid sequence of the nucleic acid molecule or a portion thereof is identified by combining data collected from at least 10 passes of the nucleic acid molecule or a portion thereof through the at least one nano-gap. In some embodiments of aspects provided herein, the nucleic acid sequence of the nucleic acid molecule or a portion thereof is identified by combining data collected from at least 20 passes of the nucleic acid molecule or a portion thereof through the at least one nano-gap. In some embodiments of aspects provided herein, the electrodes are spaced apart by a gap having a spacing from about 0.5 times to two times a molecular diameter of a given nucleic acid subunit of the nucleic acid molecule. In some embodiments of aspects provided herein, the electrodes are spaced apart by a gap having a spacing from about 0.5 times to less than a molecular diameter of a given nucleic acid subunit of the nucleic acid molecule. In some embodiments of aspects provided herein, the identifying comprises generating a consensus sequence.

Another aspect of the present disclosure provides a system for sequencing a nucleic acid molecule, comprising: (a) a chip comprising an array of individual sensors, wherein each individual sensor of the array comprises a solid state membrane having at least one nano-gap, wherein the at least one nano-gap comprises electrodes that are adapted to generate electrical current at a current level of at least about 1 picoampere to aid in detection of the nucleic acid molecule or a portion thereof upon flow of the nucleic acid molecule or a portion thereof through the at least one nano-gap at a translocation rate of at least about 0.5 kilohertz (KHz), wherein the electrodes are spaced apart by a gap having a spacing from about 0.5 times to five times a molecular diameter of a given nucleic acid subunit of the nucleic acid molecule; and (b) a computer processor coupled to the chip, wherein the computer processor is programmed to aid in characterizing a nucleic acid sequence of the nucleic acid molecule or a portion thereof by detecting the electrical current.

In some embodiments of aspects provided herein, the flow of the nucleic acid molecule or a portion thereof is facilitated without the use of a molecular motor. In some embodiments of aspects provided herein, the nucleic acid sequence of the nucleic acid molecule or a portion thereof is characterized by detecting the electrical signal at multiple time points. In some embodiments of aspects provided herein, the array of individual sensors is at a density of at least about 500 individual sensors per 1 mm². In some embodiments of aspects provided herein, the nucleic acid molecule comprises deoxyribonucleic acid (DNA) and/or ribonucleic acid (RNA). In some embodiments of aspects provided herein, the solid state membrane is manufactured from a material selected from the group consisting of a metallic material and a semiconductor material. In some embodiments of aspects provided herein, the solid state membrane is formed of a material selected from the group consisting of silicon nitrides, silica and alumina. In some embodiments of aspects provided herein, the solid state membrane has a thickness in the range of about 10 nanometers (nm) to about 1 millimeter (mm). In some embodiments of aspects provided herein, the solid state membrane has a capacitance of less than about 0.1 picofarad (pF). In some embodiments of aspects provided herein, the electrodes comprise tunneling electrodes. In some embodiments of aspects provided herein, the electrical current comprises tunneling current. In some embodiments of aspects provided herein, the nucleic acid molecule is sequenced at an accuracy of at least about 95%.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1 shows a general workflow for nucleic acid sequencing;

FIG. 2 schematically illustrates an example of test chip with sensor arrays and configuration of a sensor comprising nano-gap electrodes;

FIG. 3 shows a nucleic acid molecule passing through the nano-gap;

FIG. 4 shows a plot of signal with time measured by a sensor;

FIG. 5 schematically illustrates a computer system that is programmed or otherwise configured to implement devices, systems and methods of the present disclosure.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The term “gap,” as used herein, generally refers to a pore, channel or passage formed or otherwise provided in a material, or between electrodes. The material may be a solid state material, such as a substrate. The gap may be disposed adjacent or in proximity to a sensing circuit or an electrode coupled to a sensing circuit. In some examples, a gap has a characteristic width or diameter on the order of 0.1 nanometers (nm) to about 1000 nm. A gap having a width on the order of nanometers may be referred to as a “nano-gap” (also “nano-gap” herein). In some situations, a nano-gap has a width that is from about 0.1 nanometers (nm) to about 50 nm, 0.5 nm to 30 nm, or 0.5 nm or 10 nm, 0.5 nm to 5 nm, or 0.5 nm to 2 nm, or no greater than about 2 nm, 1 nm, 0.9 nm, 0.8 nm, 0.7 nm, 0.6 nm, or 0.5 nm. In some cases, a nano-gap has a width that is at least about 0.5 nm, 0.6 nm, 0.7 nm, 0.8 nm, 0.9 nm, 1 nm, 2 nm, 3 nm, 4 nm, or 5 nm. In some cases, the width of a nano-gap can be less than a diameter of a biomolecule or a subunit (e.g., monomer) of the biomolecule.

The term “nanopore,” as used herein, generally refers to a pore or hole having a minimum diameter on the order of nanometers and extending through a substrate. Nanopores can vary in size and can range from about 1 nanometer (nm) to about hundreds of nanometers (e.g., 300 nm, 400 nm, 500 nm, 600 nm, 700 nm, 800 nm, 900 nm) or higher in diameter. In some cases, effective nanopores have been roughly around 1.5 nm to 30 nm in diameter. The thickness of the substrate through which the nanopore extends can range from about 1 nm to about 1 micron (μm). As used herein, the terms “nano-gap” and “nanopore” are interchangeable.

The term “electrode,” as used herein, generally refers to a material or part that can be used to measure electrical current. An electrode (or electrode part) can be used to measure electrical current to or from another electrode. In some situations, electrodes can be disposed in a channel (e.g., nano-gap) and be used to measure the current across the channel. The current can be a tunneling current. Such a current can be detected upon, e.g., the flow of a biomolecule (e.g., protein) through the nano-gap, or a presence or absence of the biomolecule or a portion thereof in the nano-gap. In some cases, a sensing circuit coupled to electrodes provides an applied voltage across the electrodes to generate a current. As an alternative or in addition to, the electrodes can be used to measure and/or identify the electric conductance associated with a biomolecule (e.g., an amino acid subunit or monomer of a protein). In such a case, the tunneling current can be related to the electric conductance.

The term “biomolecule” or “biopolymer,” as used herein, generally refers to any biological material that can be interrogated with electrical parameter(s) (e.g., electrical current, voltage, differential impedance, tunneling current, resistance, capacitance, and/or conductance) across a nano-gap electrode. A biomolecule can be a nucleic acid molecule, protein, or carbohydrate. A biomolecule can include one or more subunits, such as nucleotides or amino acids.

The terms “translocation” or “translocate,” as used herein, generally refers to a movement of a biomolecule through a nano-gap or nanopore from one side of the substrate to the other. The movement can occur in a defined or a random direction. With respect to the translocation of a biomolecule through a nano-gap or nanopore, the term “in” includes the situations where the entire biomolecule is “within” and/or a portion thereof may be exterior to the nano-gap or nanopore. For instance, a biomolecule “in” the nano-gap or nanopore means that the entire biomolecule is inside the opening of the nano-gap or nanopore or only a small portion thereof is located inside the nanopore while a substantial portion is exterior to the nano-gap or nanopore.

The term “nucleic acid,” as used herein, generally refers to a molecule comprising one or more nucleic acid subunits. A nucleic acid may include one or more subunits selected from adenosine (A), cytosine (C), guanine (G), thymine (T) and uracil (U), or variants thereof, including, e.g., any naturally occurring or non-naturally occurring (e.g., modified or engineered), epigenetically modified deoxynucleotide or ribonucleotide including abasic bases. A nucleotide can include A, C, G, T or U, or variants thereof. A nucleotide can include any subunit that can be incorporated into a growing nucleic acid strand. Such subunit can be an A, C, G, T, or U, or any other subunit that is specific to one or more complementary A, C, G, T or U, or complementary to a purine (i.e., A or G, or variant thereof) or a pyrimidine (i.e., C, T or U, or variant thereof). A subunit can enable individual nucleic acid bases or groups of bases (e.g., AA, TA, AT, GC, CG, CT, TC, GT, TG, AC, CA, or uracil-counterparts thereof) to be resolved. In some examples, a nucleic acid is deoxyribonucleic acid (DNA) or ribonucleic acid (RNA), or derivatives thereof. A nucleic acid may be single-stranded or double stranded.

The term “protein,” as used herein, generally refers to a biological molecule, or macromolecule, having one or more amino acid monomers, subunits or residues. A protein containing 50 or fewer amino acids, for example, may be referred to as a “peptide.” The amino acid monomers can be selected from any naturally occurring and/or synthesized amino acid monomer, such as, for example, 20, 21, or 22 naturally occurring amino acids. In some cases, 20 amino acids are encoded in the genetic code of a subject. Some proteins may include amino acids selected from about 500 naturally and non-naturally occurring amino acids. In some situations, a protein can include one or more amino acids selected from isoleucine, leucine, lysine, methionine, phenylalanine, threonine, tryptophan and valine, arginine, histidine, alanine, asparagine, aspartic acid, cysteine, glutamine, glutamic acid, glycine, proline, serine and tyrosine.

The term “adjacent” or “adjacent to,” as used herein, includes ‘next to’, ‘adjoining’, ‘in contact with’, and ‘in proximity to’. In some instances, adjacent to components are separated from one another by one or more intervening layers. For example, the one or more intervening layers can have a thickness less than about 10 micrometers (“microns”), 1 micron, 500 nanometers (“nm”), 100 nm, 50 nm, 10 nm, 1 nm, or less. In an example, a first layer is adjacent to a second layer when the first layer is in direct contact with the second layer. In another example, a first layer is adjacent to a second layer when the first layer is separated from the second layer by a third layer.

### General Overview

The present disclosure provides devices, systems and methods for sensing or identifying biomolecules, such as, e.g., proteins, polysaccharides, lipids and nucleic acid molecules. The nucleic acid molecules can include DNA, RNA, and variants thereof. The nucleic acid molecules can be single or double stranded. The devices and systems of the present disclosure may comprise a chip having a sensor array, which may further comprise one or more nano-gap electrode pairs. Each individual nano-gap electrode pair may be configured to have a specific inter-electrode space (or nano-gap width) such that it can be used to sequence a particular type of molecules, e.g., dsDNA, or ssDNA.

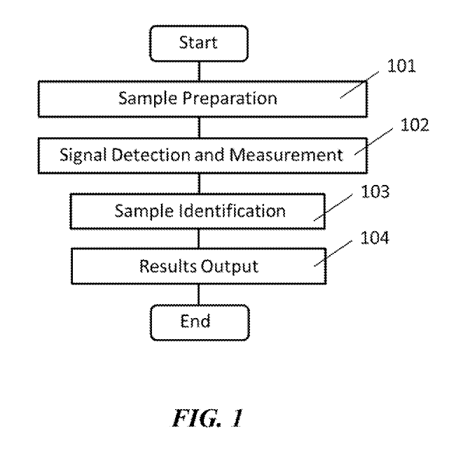

FIG. 1 shows a general workflow of the methods provided herein. In a first operation 101, a sample (e.g., a nucleic acid molecule) may be prepared for sequencing. Any substance can be the source of the sample. The substance may be a fluid, e.g., a biological fluid. A fluidic substance may include, but not limited to, blood (e.g., whole blood, plasma), cord blood, saliva, urine, sweat, serum, semen, vaginal fluid, gastric and digestive fluid, spinal fluid, placental fluid, cavity fluid, ocular fluid, serum, breast milk, lymphatic fluid, or combinations thereof.

The substance may be solid, for example, a biological tissue. The substance may comprise normal healthy tissues. The tissues may be associated with various types of organs. Non-limiting examples of organs may include brain, breast, liver, lung, kidney, prostate, ovary, spleen, lymph node (including tonsil), thyroid, pancreas, heart, skeletal muscle, intestine, larynx, esophagus, stomach, or combinations thereof.

The substance may comprise tumors. Tumors may be benign (non-cancer) or malignant (cancer). Non-limiting examples of tumors may include: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, gastrointestinal system carcinomas, colon carcinoma, pancreatic cancer, breast cancer, genitourinary system carcinomas, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, endocrine system carcinomas, testicular tumor, lung carcinoma, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, or combinations thereof. The tumors may be associated with various types of organs. Non-limiting examples of organs may include brain, breast, liver, lung, kidney, prostate, ovary, spleen, lymph node (including tonsil), thyroid, pancreas, heart, skeletal muscle, intestine, larynx, esophagus, stomach, or combinations thereof.

The sample can be isolated from the source substance with any suitable methods or techniques. In some cases, the sample is obtained by further subjecting a processed source substance (e.g., isolated nucleic acid molecule) to nucleic acid amplification conditions to generate one or more amplified products (or amplicons).

Next, once the sample has been prepared, in a second operation 102, one or more signals associated with the prepared sample may be detected and/or measured. The signals may be stored as data. For example, the prepared sample can be directed to flow through or in proximity to the one or more nano-gap electrode pairs comprised in a sensor array of a chip provided herein. The electrodes of the nano-gap electrode pair can be configured to generate a signal upon flow of each monomer or subunit of the sample molecule through the nano-gap.

Next, in a third operation 103, the detected or measured signals may be processed to aid in the determination/identification of the sample molecule, such as a sequence of the sample molecule. Next, in a fourth operation 104, results of sample determination/identification (e.g., a sequence of the sample molecule) can be outputted or delivered to a recipient (e.g., a person or an electronic system such as one or more computers and/or one or more computer servers storing and a computer-readable medium).

In some cases, one or more monomers or subunits of the sample molecule may include or be modified with labels (e.g., tunneling label, hooping label, or current blocking label). The various labels may produce the same or different signals. The labels may be able to modulate the translocation rate of the sample molecule or a portion thereof. The labels may be configured to produce distinct, detectable signals during translocation of the sample molecule through the nano-gap. In some cases, at least about 1%, 5%, 10%, 20%, 30%, 40%, 50%, 60%, 70%, 80%, 90% or more of the monomers or subunits of the sample molecule are labeled. In some cases, less than about 100%, 80%, 60%, 40%, 20%, 10%, or 5% of the monomers or subunits of the sample molecule are labeled. In cases where the monomers are modified with labels each of which produces a different signal, each of such labeled monomer or subunit can be differentially detected as the sample molecule passes through the nano-gap. As such, the sample molecule can be determined by, detecting signals or a change in the signals, which are due at least in part to the labels. Various materials including, e.g., conductors, semiconductors, magnetic materials, organic materials, inorganic materials or a combination thereof, may be employed as labels. For example, the monomers can be modified with a label that modulates resonant tunneling current including, but not limited to, metals and metal alloys and oxides thereof, e.g., gold, silver, copper, tin, titanium, iron, cobalt, chromium, molybdeneum, vanadium, aluminum, zinc, bismuth, zirconia, tungsten carbide, magnesium, cerium, of about 100 nm in diameter or less.

### Methods for Sequencing a Nucleic Acid Molecule

An aspect of the present disclosure provides a method for sequencing a nucleic acid molecule, which comprises providing a chip comprising an array of individual sensors. Each individual sensor of the array can comprise a solid state membrane having at least one nano-gap. In some cases, each individual sensor can comprise a plurality of nano-gaps. The at least one nano-gap can comprise electrodes that are adapted to generate an electrical signal to aid in detection of the nucleic acid molecule or a portion thereof upon flow of the nucleic acid molecule or a portion thereof through the at least one nano-gap.

Subsequently, the nucleic acid molecule or a portion thereof can be directed through or in proximity to the at least one nano-gap. For example, the nucleic acid molecule can be subjected to a flow through the nano-gap such that individual subunits of the nucleic acid molecule can flow through the nano-gap.

Next, a nucleic acid sequence of the nucleic acid molecule or a portion thereof can be identified. The nucleic acid sequence can be identified at an accuracy of at least about 60%, 70%, 80%, 85%, 90%, 95%, 95.5%, 96%, 96.5%, 97%, 97.5%, 98%, 98.5%, 99%, 99.5%, 99.9%, or 99.99%. In some cases, the nucleic acid sequence can be determined/identified at accuracy between any of the two values described herein, for example, about 94%. In some cases, a predetermined accuracy is specified by a user, and a system may modify one or more parameters, e.g., the number of passes of the molecule or a portion thereof through the nano-gap, the translocation rate of the molecule, the voltage applied to the electrodes, or length of interrogation, to reach the desired or predetermined accuracy.

Additionally or alternatively, in another aspect of the present disclosure, a method for sequencing a nucleic acid molecule can comprise activating a chip which comprises an array of individual sensors. Each individual sensor of the array can comprise a solid state membrane having at least one nano-gap. The at least one nano-gap can comprise electrodes that are adapted to generate an electrical signal to aid in detection of the nucleic acid molecule or a portion thereof upon flow of the nucleic acid molecule or a portion thereof through the at least one nano-gap. The electrodes can be spaced apart by a gap of at least about 0.01 times, 0.05 times, 0.1 times, 0.2 times, 0.3 times, 0.4 times, 0.5 times, 0.6 times, 0.7 times, 0.8 times, 0.9 times, 1 time, 1.5 times, 2 times, 2.5 times, 3 times, 3.5 times, 4 times, 4.5 times, or 5 times a molecular diameter of a given nucleic acid subunit of the nucleic acid molecule. In some cases, the electrodes can be spaced apart by a gap less than or equal to about 100 times, 90 times, 80 times, 70 times, 60 times, 50 times, 40 times, 30 times, 20 times, 15 times, 10 times, 9 times, 8 times, 7 times, 6 times, 5 times, 4 times, 3 times, 2 times, 1 times, 0.8 times, 0.6 times, 0.4 times, 0.2 times or 0.1 times a molecular diameter of a given nucleic acid subunit of the nucleic acid molecule. In some cases, the electrodes can be spaced apart by a gap falling between any of the two values described herein, for example, from about 0.5 times to five times a molecular diameter of a given nucleic acid subunit of the nucleic acid molecule.

Next, the nucleic acid molecule or a portion thereof can be directed through or in proximity to the at least one nano-gap and a nucleic acid sequence of the nucleic acid molecule or a portion thereof can be identified based upon the generated electrical signal(s).

The nucleic acid sequence can be identified at an accuracy of at least about 60%, 70%, 80%, 85%, 90%, 95%, 95.5%, 96%, 96.5%, 97%, 97.5%, 98%, 98.5%, 99%, 99.5%, 99.9%, or 99.99%. In some cases, the nucleic acid sequence can be determined/identified at accuracy between any of the two values described herein. In some examples, the nucleic acid sequence of said nucleic acid molecule or portion thereof is determined at an accuracy of at least about 60%, 70%, 80%, 85%, 90%, 95%, 95.5%, 96%, 96.5%, 97%, 97.5%, 98%, 98.5%, 99%, 99.5%, 99.9%, or 99.99% over a span of at least about 5, 10, 20, 30, 40, 50, 60, 70, 80, 90, 100, 200, 300, 400, 500, 600, 700, 800, 900, 1,000, 2,000 or 3,000 contiguous nucleic acid bases of the nucleic acid molecule.

In some cases, high accuracy is achieved in the absence of re-sequencing the nucleic acid molecule or a portion thereof. In some cases, the high-accuracy identification of the nucleic acid sequence is achieved by performing multiple passes (i.e., sequencing a nucleic acid molecule or a portion thereof multiple times, e.g., by passing the nucleic acid molecule or a portion thereof through at least one set of electrodes several times and determining the nucleic acid sequence each time the molecule or a portion thereof passes through the at least one nano-gap of the electrode pair). The number of passes can be any number, integer or non-integer. In some cases, the nucleic acid sequence is identified at a high accuracy by sequencing the same nucleic acid molecule or a portion thereof at least about 2 times, 3 times, 4 times, 5 times, 6 times, 7 times, 8 times, 9 times, 10 times, 12 times, 14 time, 16 times, 18 times, 20 times, 25 times, 30 times, 35 times, 40 times, 45 times, 50 times, 60 time, 70 times, 80 times, 90 times, 100 times, 250 times, 500 times, or more. In some cases, the nucleic acid molecule or a portion thereof is sequenced at most 1000 times, 750 times, 500 times, 250 times, 100 times, 75 times, 50 times, 40 times, 30 times, 25 times, 20 times, 15 times, 10 times, 9 times, 8 times, 7 times, 6 times, 5 times, 4 times, 3 times, 2 times, 1 time, or less. In some cases, the number of passes or multiple sequencing falls between any of the two values described herein, for example, 11 times.

In cases where multiple passes are utilized, multiple passes can occur upon a single pair (or set) of electrodes or a plurality of sets of electrodes, and data from multiple passes can be combined. For example, the nucleic acid sequence can be determined at a high accuracy by combining data collected from a total of about 5 passes, 10 passes, 50 passes, or any number of passes in between. In some examples, the high accuracy (e.g., at least about 97%) is achieved by combining data collected from at most about 100 passes, 80 passes, 60 passes, 50 passes, 40 passes, 30 passes, 20 passes, 10 passes, 9 passes, 8 passes, 7 passes, 6 passes, 5 passes, 4 passes, 3 passes, 2 passes, or a single pass. In some cases, multiple passes or binding is combined within single data acquisition.

Upon identifying the nucleic acid sequence, a consensus sequence may be generated. The consensus sequence can be generated upon alignment of multiple sequencing reads. The consensus sequence can be generated by sequencing and resequencing the nucleic acid molecule one or more times. The same sequence of the nucleic acid molecule can be sequenced multiple times.

In some situations, one or more groups of nucleic acid bases are identified using the methods of the present disclosure. For example, a combination of three nucleic acid bases are determined by their characteristic effect on the electrical signal generated in the nano-gap. In such cases, the high accuracy (e.g., at least about 90%, 95%, 97%, or 99%) is achieved when identifying greater than or equal to about 1, 2, 3, 4, 5, 6, 7, or 8 nucleic acid bases (i.e., as a group). Alternatively or additionally, the accuracy of nucleic acid sequencing is high when identifying up to about 10, 9, 8, 7, 6, 5, 4, 3, or 2 nucleic acid bases. In some cases, the accuracy is high (e.g., at least about 90%, 95%, 97%, or 99%) when identifying a single nucleic acid base of the nucleic acid molecule or a portion thereof.

As described above and elsewhere herein, the nucleic acid molecule can be from various sources, for example, a biological sample containing one or more nucleic acid molecules. The biological sample can be obtained (e.g., extracted or isolated) from a bodily sample of a subject, for example, a bodily fluid. The bodily sample can be selected from blood (e.g., whole blood), plasma, serum, urine, saliva, mucosal excretions, sputum, stool and tears. The bodily sample can be a fluid or tissue sample (e.g., skin sample) of the subject. In some examples, the sample is obtained from a cell-free bodily fluid of the subject, such as whole blood. In such instance, the sample can include cell-free DNA and/or cell-free RNA. In some other examples, the sample is an environmental sample (e.g., soil, waste, ambient air and etc.), industrial sample (e.g., samples from any industrial processes), and food samples (e.g., dairy products, vegetable products, and meat products).

The membrane can be a device. For example, the membrane is a solid state device having the at least one nano-gap. The membrane can be formed of multiple solid state subunits. The membrane can be formed (e.g., at least partially) of various materials, e.g., biological, non-biological, organic, inorganic, semiconducting, insulating, magnetic, or metallic materials. Non-limiting examples of materials may include carbon, silica, silicon, alumina, plastic, glass, metal, metal-alloy, polymer, nylon, polymerized Langmuir Blodgett film, functionalized glass, Ge, GaAs, Gap, SiN₄, modified silicon, or any one of a variety of gels or polymers such as (poly)tetrafluoroethylene, (poly)vinylidenedifluoride, polystyrene, polycarbonate, or combinations thereof. The membrane can exist as a form of particles, strands, precipitates, gels, sheets, tubing, spheres, containers, capillaries, pads, slices, films, plates, slides, etc. The membrane can take on any surface configurations (e.g., planar or non-planar). For example, the substrate may contain raised or depressed regions on which fabrication or deposition of at least a pair of electrodes may take place.

Thickness of the solid state membrane may vary. In some cases, the solid state membrane may have a thickness of greater than or equal to about 0.01 nanometers (nm), 0.05 nm, 0.075 nm, 0.1 nm, 0.25 nm, 0.5 nm, 0.75 nm, 1 nm, 2.5 nm, 5 nm, 7.5 nm, 10 nm, 25 nm, 50 nm, 75 nm, 100 nm, 250 nm, 500 nm, 750 nm, 1 micrometer (μm), 5 μm, 10 μm, 25 μm, 50 μm, 75 μm, 100 μm, 250 μm, 500 μm, 750 μm, 1 millimeter (mm), 5 mm, 10 mm, 20 mm, 30 mm, 40 mm, 50 mm, or more. In some cases, the thickness of the solid state membrane can be smaller than or equal to about 100 mm, 50 mm, 25 mm, 10 mm, 5 mm, 1 mm, 900 μm, 800 μm, 700 μm, 600 μm, 500 μm, 400 μm, 300 μm, 200 μm, 100 μm, 80 μm, 60 μm, 40 μm, 20 μm, 10 μm, 9 μm, 8 μm, 7 μm, 6 μm, 5 μm, 4 μm, 3 μm, 2 μm, 1 μm, 800 nm, 600 nm, 400 nm, 200 nm, 100 nm, 80 nm, 60 nm, 40 nm, 20 nm, 10 nm, 5 nm, 1 nm, 0.5 nm, 0.1 nm, or less. In some cases, the thickness of the solid state membrane can be between any of the two values described above, e.g., from about 10 nm to about 1 mm.

The membrane can be functional, which may have certain electrical properties, such as resistance, capacitance and/or conductance. For example, the membrane can have a capacitance that is less than or equal to about 10 picofarad (pF), 9 pF, 8 pF, 7 pF, 6 pF, 5 pF, 4 pF, 3 pF, 2 pF, 1 pF, 0.9 pF, 0.8 pF, 0.7 pF, 0.6 pF, 0.5 pF, 0.4 pF, 0.3 pF, 0.2 pF, 0.1 pF, 0.075 pF, 0.05 pF, 0.025 pF, 0.01 pF, 0.005 pF, 0.001 pF or between any of the two values described herein.

Further, the membrane can have a certain inter-electrode capacitance (i.e., capacitance between electrodes). In some cases, the inter-electrode capacitance can be greater than or equal to about 0.1 femtofarad (fF), 0.25 fF, 0.5 fF, 0.75 fF, 1 fF, 2.5 fF, 5 fF, 7.5 fF, 10 fF, 20 fF, 30 fF, 40 fF, 50 fF, 60 fF, 70 fF, 80 fF, 90 fF, 100 fF, 200 fF, 300 fF, 400 fF, 500 fF, 600 fF, 700 fF, 800 fF, 900 fF, 1,000 fF, or more. In some cases, the inter-electrode capacitance can be less than or equal to about 2000 fF, 1500 fF, 1000 fF, 800 fF, 600 fF, 400 fF, 200 fF, 100 fF, 80 fF, 60 fF, 40 fF, 20 fF, 10 fF, 9 fF, 8 fF, 7 fF, 6 fF, 5 fF, 4 fF, 3 fF, 2 fF, 1 fF, 0.5 fF, 0.1 fF or less.

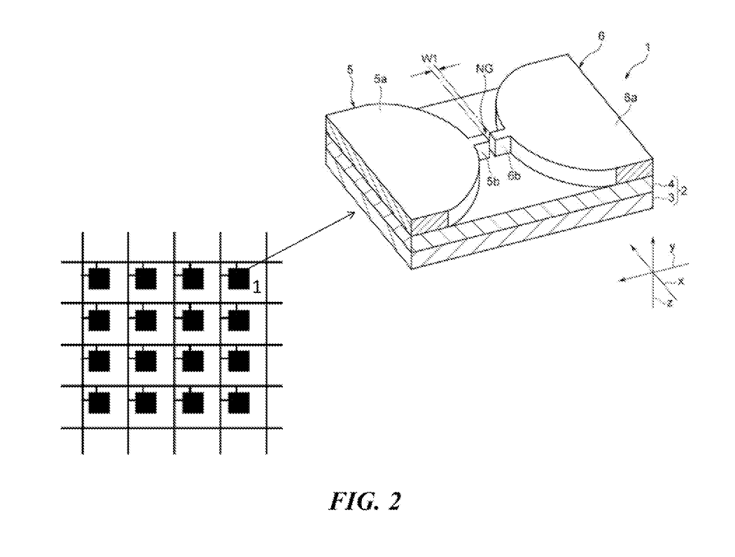

FIG. 2 shows an example chip comprising an n×m (e.g., 4×4) senor array, wherein ‘n’ and ‘m’ may be integers greater than or equal to 1, as well as the configuration of an exemplary nano-gap electrode pair 1 comprised in each sensor. As illustrated in FIG. 2, for the nano-gap electrode pair 1, opposing electrodes 5 and 6 are disposed on a substrate 2. A nano-gap NG with a width W1 which is of nanoscale (no larger than, for example, 1000 nanometers) is formed between electrodes 5 and 6. The width W1 may be between 0.1 nanometers (nm) and 1,000 nm, or no greater than or equal to about 500 nm, 400 nm, 300 nm, 200 nm, 100 nm, 80 nm, 60 nm, 40 nm, 20 nm, 10 nm, 9 nm, 8 nm, 7 nm, 6 nm, 5 nm, 4 nm, 3 nm, 2 nm, 1 nm, 0.9 nm, 0.8 nm, 0.7 nm, 0.6 nm, or 0.5 nm, or of any other widths as described herein. In some cases, the electrodes are spaced apart with a nano-gap width greater than or equal to about 0.1, 0.2, 0.3, 0.4, 0.5, 0.6, 0.7, 0.8, 0.9, 1, 2, 3, 4, 5, 6, 7, 8, 9, 10, 15, 20, 40, 60, 80, or 100 times a molecular diameter of a given nucleic acid subunit (or combination of nucleic acid subunits) of the nucleic acid molecule. In some cases, the electrodes are spaced apart such that the nano-gap width is less than or equal to about 20, 15, 10, 8, 6, 4, 2, 1, 0.5, 0.25, or 0.1 times a molecular diameter of a given nucleic acid subunit (or combination of nucleic acid subunits) of the nucleic acid molecule. In some cases, the the nano-gap width falls into a range between any of the two value described above, e.g., from about 0.5 times to about five times, from about 0.5 times to about two times, or from about 0.5 times to less than or equal to one molecular diameter of a given nucleic acid subunit.

Substrate 2 may be composed of, for example, a silicon substrate 3 and a silicon oxide layer 4 formed thereon. As an alternative, substrate 2 may include other semiconductor materials(s), including a Group IV or Group III-V semiconductor, such as germanium or gallium arsenide, including oxides thereof. Substrate 2 can have a configuration in which two electrodes 5 and 6 forming a pair may be formed on silicon oxide layer 4. Each of the electrodes 5 and 6 may be formed from material including, e.g., metal and metal silicides, and in some cases may be formed almost bilaterally symmetrically across nano-gap NG on substrate 2. Non-limiting examples of electrode-forming materials may include platinum, copper, silver, gold, alloy, titanium nitride (TiN), titanium silicide, molybdenum silicide, platinum silicide, nickel silicide, cobalt silicide, palladium silicide, niobium silicide, alloys of silicide with other materials (e.g., carbon nanotubes or graphene etc.), silicides doped with various materials suitable for doping of semiconductors, or combinations thereof.

In some cases, electrodes 5 and 6 have substantially the same configuration and may be composed of leading electrode edges 5b and 6b forming nano-gap NG, and base parts 5a and 6a may be integrally formed with the root portions of the leading electrode edges 5b and 6b. Leading electrode edges 5b and 6b may comprise, for example, rectangular solids, the longitudinal directions of which may extend in a y-direction, and may be disposed so that the apical surfaces of the leading electrode edges 5b and 6b face each other; leading edges 5b and 6b may have curves (not shown).

Base parts 5a and 6a may have protrusions at the central apical ends thereof whereby the leading electrode edges 5b and 6b may be formed. A gently curved surface may be formed toward both sides of each base part 5a and 6a with the central apical end thereof at the center. Thus, base parts 5a and 6a may be formed into a curved shape with leading electrode edges 5b and 6b positioned at the vertexes. Note that electrodes 5 and 6 may be configured so that when a solution containing single-stranded DNA, for example, is supplied from an x-direction orthogonal to the y-direction which may be the longitudinal direction of electrodes 5 and 6 and to a z-direction which may be the vertical direction of electrodes 5 and 6 and may intersect at right angles with this y-direction, the solution may be guided along the curved surfaces of base parts 5a and 6a to leading electrode edges 5b and 6b to enable the solution to reliably pass through nano-gap NG.

Further, for a nano-gap electrode pair 1 configured as described above, current can be supplied from, for example, a power source (not shown) to electrodes 5 and 6, and values of current flowing across electrodes 5 and 6 can be measured with an ammeter (not shown). Accordingly, a nano-gap electrode pair 1 allows e.g., a nucleic acid molecule such as a single-stranded DNA to pass through a nano-gap NG between electrodes 5 and 6 from the x-direction; an ammeter to measure values of currents flowing across electrodes 5 and 6 when bases (e.g., modified bases) of the single-stranded DNA passes through nano-gap NG between electrodes 5 and 6; and the bases constituting the single-stranded DNA may be determined on the basis of the correlated current values.

As described above and elsewhere herein, each individual sensor of the sensor array may comprise one or more nano-gap electrode pairs with the same or differing configurations (e.g., gap width W1, shape of electrodes, inter-electrode distance, substrate materials and electrode materials etc.), depending upon, the applications. The design, fabrication, configuration, and applications of nano-gap electrode may be as described in, for example, PCT Patent Publication No. WO/2015/057870, and PCT Patent Publication No. WO/2015/028886, each of which is incorporated herein by reference in its entirety.

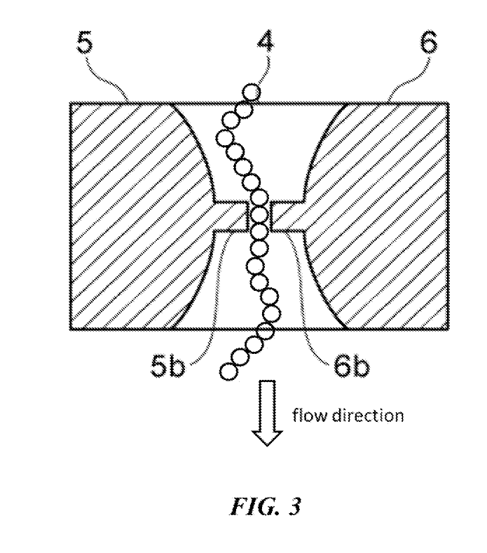

FIG. 3 schematically illustrates the detection and/or identification of nucleic acid sequence using a sensor having nano-gap electrode while the nucleic acid molecule passing through the nano-gap. With reference to FIG. 3, the nucleic acid bases of a single-stranded nucleic acid molecule are directed to pass through the nano-gap between electrodes 5 and 6 at the x-direction (shown in FIG. 2). The flow or movement of the nucleic acid molecule through the nano-gap can be facilitated with or without the aid of molecular motors (e.g., enzyme), or upon the application of electrical stimulus (e.g., an electrical potential (V) between the electrodes, which can provide an electric field that directs the flow of nucleic acid molecule along a certain direction). In cases where the flow of the nucleic acid molecule through the nano-gap is facilitated with molecular motors, the molecular motors can be biological molecular machines that are the essential agents of movement in living organisms, or devices that consume energy in one form and convert it into motion or mechanical work. For example, many protein-based molecular motors harness the chemical free energy released by the hydrolysis of ATP in order to perform mechanical work. Non-limiting examples of molecular motors include RNA polymerase, DNA polymerase, RNA dependent polymerase, DNA dependent polymerase, helicases, topoisomerases, chromatin structure remodeling (RSC) complex, nucleosome remodeling complex such as SWI/SNF, structural maintenance of chromosomes (SMC) proteins, viral DNA packaging motors, synthetic molecular motors, proteins that condense chromosome, and the like.

Translocation rate at which the nucleic acid molecule passes through the at least one nano-gap can vary, depending upon, for example, level of generated electrical signal, level of background signal, size, shape, structure, and/or composition of the nucleic acid molecule, configuration of nano-gap electrode pair, presence or absence of molecular motors, presence or absence of external stimulus (e.g., electrical potential), and/or desired signal-to-noise ratio (S/N ratio). For example, the nucleic acid molecule can be directed through the at least one nano-gap at a translocation rate of at least about 0.1, 0.2, 0.3, 0.4, 0.5, 0.6, 0.7, 0.8, 0.9, 1, 2, 3, 4, or 5 KHz at a current level of at least about 0.1, 0.5, 1, 5, 10, 50, 100, 200, 300, 400, 500, 600, 700, 800, 900, 1,000 picoampere (pA), 1.5, 2, 2.5, 3, 3.5, 4, 4.5, or 5 nanoampere (nA), a background current level of equal to or less than about 5, 4.5, 4, 3.5, 3, 2.5, 2, 1.5, 1, 0.9, 0.8, 0.7, 0.6, 0.5, 0.4, 0.3, 0.2, or 0.1 nA, and a signal to noise ratio of at least about 1, 2, 3, 4, 5, 6, 7, 8, 9, 10 or more.

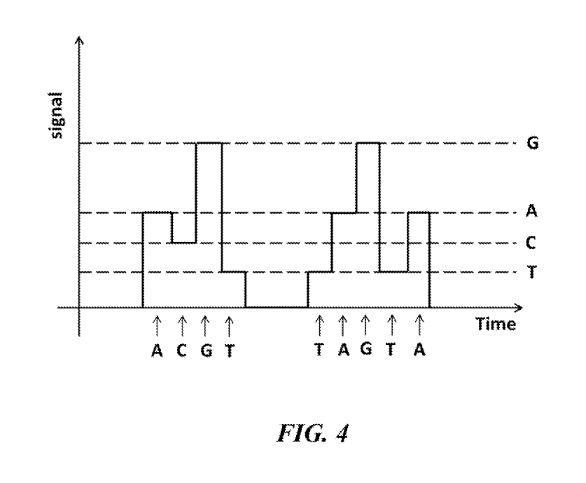

The two electrodes of a nano-gap electrode pair may be spaced apart to have a nano-gap width W1 such that a single electrical signal is generated each time when a monomer (i.e., each nucleic acid base of the nucleic acid molecule to be sequenced) or a combination of nucleic acid subunits (e.g., a group of nucleic acid bases such as adenine, cytosine, and thymine) passes through the nano-gap. The generated signal or change thereof can then be detected (or measured) and the nucleic acid sequence can be identified based upon the detected signal corresponding to each type of monomer or subunit (shown in FIG. 4). In some cases, detecting or measuring electrical signals can comprise measuring the change of an electrical signal of a nucleic acid molecule relative to one or more reference molecules, and the relative values between the electrical signal and reference signal(s) can be used to determine the nucleic acid sequence of the nucleic acid molecule.

The signals can be any types of electrical signals generated upon the passage of the nucleic acid molecule through the one or more nano-gaps on each individual sensor, e.g., voltage, current, conductance etc. The electrical signals can comprise tunneling current when tunneling electrodes are utilized, and a measurement device can be employed for measuring tunneling current generated upon the passage of nucleic acid subunits through the nano-gap(s). In some cases, a measurement device (or measurement unit) may be provided to measure the signal. The measurement device may comprise an ammeter, a current mirror, or any other current measurement or amplification approach, and an approach for quantifying the current, which may include an analog to digital converter (ADC), a delta sigma ADC, a flash ADC, a dual slope ADC, a successive approximation ADC, an integrating ADC, or any other appropriate type of ADC. The ADC may have a linear relationship between its output and the input, or may have an output which is tuned to the particular current levels which may be expected for a particular combination of bases, modified bases expected and metals utilized in a nano-gap electrode pair. The response may be fixed, or may be adjustable, and may be adjustable particularly in conjunction with different outputs associated with the different nucleobases and or nucleobases modifications which may be utilized.

Further, based on measured tunneling current, a conductance may be obtained so as to create a conductance-time profile which may then be used for determining or identifying the nucleic acid sequence. Conductance can be calculated by dividing values of tunneling current by a voltage V applied to the electrodes of the nano-gap electrode pair. With the use of conductance, even when the applied voltage varies or fluctuates among measurements, profiles with a unified reference may be obtained.

Additionally or alternatively, a current amplifier can be used to amplify current measured by ammeter. Such current amplifier can be off the chip, partially embedded, or completely embedded in the chip. Use of a current amplifier, a value of measured current can be amplified, and the signal can be measured with a higher sensitivity and accuracy.

In some situations, the signals are at least partially obscured by noise signal. The signal to noise (S/N) ratio can be any suitable high value that can identify nucleic acid sequence at a high accuracy. In some cases, the one or more nucleic acid subunits can be detected at a S/N ratio greater than or equal to about 1-to-1, 2-to-1, 3-to-1, 4-to-1, 5-to-1, 6-to-1, 7-to-1, 8-to-1, 9-to-1, 10-to-1, 20-to-1, 50-to-1, 75-to-1, 100-to-1, 250-to-1, 500-to-1, 750-to-1, 1000-to-1, 10000-to-1, or more. In some cases, the one or more nucleic acid subunits can be detected at a S/N ratio that is between any of the two values described above, for example, from about 1 to 2.

Although a time utilized for measuring an electrical signal or determining an individual nucleic acid subunit is not limited, the time period utilized may be less than or equal to about 1 minute (min), 50 seconds (s), 40 s, 30 s, 20 s, 10 s, 5 s, 1 s, 800 millisecond (ms), 600 ms, 400 ms, 200 ms, 100 ms, 80 ms, 60 ms, 40 ms, 20 ms, 10 ms, 9 ms, 8 ms, 7 ms, 6 ms, 5 ms, 4 ms, 3 ms, 2 ms, 1 ms, 900 microseconds (μs), 800 μs, 700 μs, 600 μs, 500 μs, 400 μs, 300 μs, 200 μs, 100 μs, 80 μs, 60 μs, 40 μs, 20 μs, 10 μs, 5 μs, 1 μs, or less. In some cases, the time period for detecting individual nucleic acid subunits is at least about 0.1 μs, 0.5 μs, 1 μs, 10 μs, 50 μs, 100 μs, 250 μs, 500 μs, 750 μs, 1 ms, 5 ms, 10 ms, 25 ms, 50 ms, 75 ms, 100 ms, 250 ms, 500 ms, 750 ms, 1 s or more. In some cases, the time period for detecting individual nucleic acid subunits is between any of the two values described above, for example, from 1 μs to 1 ms.

Nucleic acid sequence of the nucleic acid molecule or a portion thereof can be identified at a certain frequency (i.e., sequence detection rate). In some cases, the nucleic acid sequence can be identified at a frequency less than or equal to about 500 KHz (1/second), 400 KHz, 300 KHz, 200 KHz, 150 KHz, 100 KHz, 80 KHz, 60 KHz, 40 KHz, 20 KHz, 10 KHz, 5 KHz, 1 KHz, 0.9 KHz, 0.8 KHz, 0.7 KHz, 0.6 KHz, 0.5 KHz, 0.4 KHz, 0.3 KHz, 0.2 KHz, 0.1 KHz, 0.05 KHz, 0.01 KHz, or less. In some cases, the nucleic sequence can be identified at a frequency greater than or equal to about 0.001 KHz, 0.01 0.1 KHz, 0.5 KHz, 1 KHz, 10 KHz, 30 KHz, 50 KHz, 70 KHz, 90 KHz, 100 KHz, 200 KHz, 250 KHz, 300 KHz, or more. In some cases, the nucleic sequence can be identified at a frequency in between any of the two values described above, for example, from about 0.1 KHz to about 100 KHz.

### Systems for Sequencing a Nucleic Acid Molecule

In another aspect of the present disclosure, a system for sequencing a nucleic acid molecule comprises a chip comprising an array of individual sensors. Each individual sensor of the array can comprise a solid state membrane configured to have at least one nano-gap therein. The at least one nano-gap can comprise electrodes coupled to an electrical circuit that is adapted to generate electrical signals to aid in detection of the nucleic acid molecule or a portion thereof upon flow of the nucleic acid molecule or a portion thereof through the at least one nano-gap.

The system of the present disclosure can further comprise a computer processor coupled to the chip. The computer processor can be programmed to aid in characterizing a nucleic acid sequence of the nucleic acid molecule or the portion thereof based on the electrical signals received from the array of individual sensors. The electrical signals can comprise any signals that are measurable, such as voltage, current, conductance, or resistance. The electrical signals can be detected at multiple time points or monitored in real-time. With detected signals, signal-time profile can be generated which may be used for determining or identifying the nucleic acid sequence.

The nucleic acid sequence of the nucleic acid molecule or the portion thereof can be characterized at an accuracy of at least about 60%, 70%, 80%, 85%, 90%, 95%, 95.5%, 96%, 96.5%, 97%, 97.5%, 98%, 98.5%, 99%, 99.5%, 99.9%, 99.99%, or greater. Such high accuracy can be achieved in the absence of re-sequencing the nucleic acid molecule or a portion thereof. In some cases, the high accuracy is achieved by performing a single pass of the nucleic acid molecule or a portion thereof. In some cases, the high accuracy is achieved by performing multiple passes (i.e., sequencing a nucleic acid molecule a plurality of times e.g., by passing the nucleic acid molecule through or in proximity to one or more nano-gaps multiple times and sequencing nucleic acid bases of the nucleic acid molecule) and combining data collected from some of or all the passes.

The electrodes can be coupled to an electric circuit, which is configured to apply voltage across the electrodes of a nano-gap electrode pair. In some cases, voltage across different nano-gap electrode pairs may be different, and may particularly be different as a function of a nano-gap spacing associated with a particular electrode pair. In some situations, the sensors are coupled to an integrated circuit that measures, collects and processes the electrical signals. In some cases, the sensors are coupled to a plurality of integrated sensors and each sensor is associated with an individual integrated circuit which is independently addressable (i.e., each integrated circuit is configured to independently control, send signals to, and collect data from the associated sensor). In some cases, the sensors are sorted into different groups and each group of sensors is connected to an integrated circuit that is independently addressable. The integrated circuit(s) can be part of the chip. In some situations, the sensors can be part of the chip. In some cases, the system may further comprise a transimpedence amplifier. The transimpedence amplifier can be operatively coupled to the computer processor. In some cases, the system may further comprise a charge sensitive amplifier, used in either discrete time or continuous mode.

As will be appreciated, in some cases, it can be advantageous to provide a chip having a sensor array with a high density (i.e., number of individual sensors per unit area) of individual sensors. For example, a chip with high density of individual sensors may facilitate the fabrication of devices with smaller footprint, which are portable and cost less. With a given surface area, a chip with high density of individual sensors allows for high through-put and/or low-cost sequencing (i.e., a larger number of nucleic acid molecules to be sequenced in parallel).

The chip can comprise a sensor array at any density which may be suitable (e.g., a density suitable for nucleic sequencing at a per-determined sensitivity and/or accuracy). In some cases, the sensor array comprises individual sensors at a density greater than or equal to about 10, 50, 100, 200, 300, 400, 500, 600, 700, 800, 900, 1,000, 2,000, 3,000, 4,000, 5,000, 6,000, 7,000, 8,000, 9,000, 10,000, 20,000, 30,000, 40,000, 50,000, 60,000, 70,000, 80,000, 90,000, 100,000, 200,000, 300,000, 400,000, 500,000, 750,000, 1,000,000, 2,000,000, 3,000,000, 4,000,000, 5,000,000, 6,000,000, 7,000,000, 8,000,000, 9,000,000, 10,000,000, 20,000,000, 30,000,000, 40,000,000, 50,000,000, 60,000,000, 70,000,000, 80,000,000, 90,000,000, 100,000,000, 200,000,000, 300,000,000, 400,000,000, or 500,000,000 individual sensors/mm², or more. In some cases, the sensor array comprises individual sensors at a density less than or equal to about 1,000,000,000, 800,000,000, 600,000,000, 400,000,000, 100,000,000, 80,000,000, 60,000,000, 40,000,000, 10,000,000, 8,000,000, 6,000,000, 4,000,000, 2,000,000, 1,000,000, 800,000, 600,000, 400,000, 200,000, 100,000, 80,000, 60,000, 40,000, 20,000, 10,000, 8,000, 6,000, 5,000, 4,000, 2,000, 1,000, 800, 600, 400, 200, 100, 50 individual sensors/mm² or less. In some cases, the density of individual sensors falls between any of the two values described above, for example, at about 5,500, 37,500, or 250,000 individual sensors/mm².

The sensors can be independently or individually addressable. Independently or individually addressable sensors can be controlled (e.g., by the applied bias voltage), addressed, processed, and/or have data read individually or separately. Alternatively, the individual sensors can be sorted into different groups and each group of sensors can be independently addressed. Sensors comprised in each group may or may not be the same. Each group of sensors may comprise, for example, greater than or equal to about 1, 5, 10, 25, 50, 75, 100, 200, 400, 600, 800, 1,000, 2,000, 3,000, 4,000, or 5,000 sensors. In some cases, each group of sensors may comprise less than or equal to about 50,000, 25,000, 10,000, 8,000, 6,000, 4,000, 2,000, 1,000, 750, 500, 250, 100, 75, 50, 25, or 10 sensors. In some cases, the number of sensors comprised in each group may be between any of the two values described above, e.g., between 5 and 500 sensors per group.

### Computer Systems

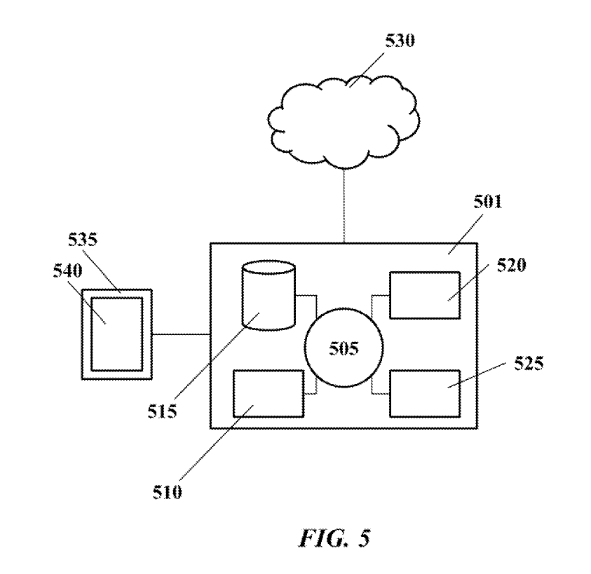

The present disclosure provides computer control systems that are programmed or otherwise configured to implement methods provided herein, such as calibrating sensors of the present disclosure. FIG. 5 shows a computer system 501 that includes a central processing unit (CPU, also “processor” and “computer processor” herein) 505, which can be a single core or multi core processor, or a plurality of processors for parallel processing. The computer system 501 also includes memory or memory location 510 (e.g., random-access memory, read-only memory, flash memory), electronic storage unit 515 (e.g., hard disk), communication interface 520 (e.g., network adapter) for communicating with one or more other systems, and peripheral devices 525, such as cache, other memory, data storage and/or electronic display adapters. The memory 510, storage unit 515, interface 520 and peripheral devices 525 are in communication with the CPU 505 through a communication bus (solid lines), such as a motherboard. The storage unit 515 can be a data storage unit (or data repository) for storing data. The computer system 501 can be operatively coupled to a computer network (“network”) 530 with the aid of the communication interface 520. The network 530 can be the Internet, an internet and/or extranet, or an intranet and/or extranet that is in communication with the Internet. The network 530 in some cases is a telecommunication and/or data network. The network 530 can include one or more computer servers, which can enable distributed computing, such as cloud computing. The network 530, in some cases with the aid of the computer system 501, can implement a peer-to-peer network, which may enable devices coupled to the computer system 501 to behave as a client or a server.

The CPU 505 can execute a sequence of machine-readable instructions, which can be embodied in a program or software. The instructions may be stored in a memory location, such as the memory 510. The instructions can be directed to the CPU 505, which can subsequently program or otherwise configure the CPU 505 to implement methods of the present disclosure. Examples of operations performed by the CPU 505 can include fetch, decode, execute, and writeback.

The CPU 505 can be part of a circuit, such as an integrated circuit. One or more other components of the system 501 can be included in the circuit. In some cases, the circuit is an application specific integrated circuit (ASIC).

The storage unit 515 can store files, such as drivers, libraries and saved programs. The storage unit 515 can store user data, e.g., user preferences and user programs. The computer system 501 in some cases can include one or more additional data storage units that are external to the computer system 501, such as located on a remote server that is in communication with the computer system 501 through an intranet or the Internet. The computer system 501 can communicate with one or more remote computer systems through the network 530.

Methods as described herein can be implemented by way of machine (e.g., computer processor) executable code stored on an electronic storage location of the computer system 501, such as, for example, on the memory 510 or electronic storage unit 515. The machine executable or machine readable code can be provided in the form of software. During use, the code can be executed by the processor 505. In some cases, the code can be retrieved from the storage unit 515 and stored on the memory 510 for ready access by the processor 505. In some situations, the electronic storage unit 515 can be precluded, and machine-executable instructions are stored on memory 510.

The code can be pre-compiled and configured for use with a machine having a processor adapted to execute the code, or can be compiled during runtime. The code can be supplied in a programming language that can be selected to enable the code to execute in a pre-compiled or as-compiled fashion.

The computer system 501 can be programmed or otherwise configured to regulate one or more parameters, such as the voltage applied across electrodes of a nano-gap electrode pair, temperature, flow rate of nucleic acid molecules, and time period for signal acquisition.

Aspects of the systems and methods provided herein, such as the computer system 501, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 501 can include or be in communication with an electronic display 535 that comprises a user interface (UI) 540 for providing, for example, signals from a chip with time. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 505.

Devices, systems and methods of the present disclosure may be combined with and/or modified by other devices, systems, or methods, such as those described in, for example, PCT Patent Publication No. WO/2015/057870, and PCT Patent Publication No. WO/2015/028886, each of which is entirely incorporated herein by reference.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-113. (canceled)

114. A method for sensing a sample molecule using a nano-gap sensor, comprising:
(a) providing a chip comprising said nano-gap sensor, said nano-gap sensor comprising a nano-gap comprising one or more nano-gap electrode pairs that are adapted to generate signals upon flow of electrical current across said nano-gap;
(b) flowing said sample molecule or portion thereof through or in proximity to said nano-gap, wherein said sample molecule comprises a plurality of monomers comprising labels that permit individual monomers of said plurality of monomers to be identified from one another upon flow of said sample molecule or portion thereof through or in proximity to said nano-gap;
(c) using said one or more electrode pairs to detect said signals or changes thereof based at least in part on said labels while said sample molecule or portion thereof flows through or in proximity to said nano-gap; and
(d) using said signals or changes thereof to identify said individual monomers, thereby determining a sequence of said sample molecule or portion thereof.

115. The method of claim 114, wherein said sample molecule is a nucleic acid molecule.

116. The method of claim 115, wherein said sequence of said nucleic acid molecule or portion thereof is determined at an accuracy of at least about 97% over a span of at least about 100 contiguous nucleic acid bases of said nucleic acid molecule.

117. The method of claim 115, wherein said sequence of said nucleic acid molecule or a portion thereof is determined at an accuracy of at least about 97% in the absence of re-sequencing said nucleic acid molecule or a portion thereof.

118. The method of claim 115, wherein said nucleic molecule is double stranded.

119. The method of claim 118, wherein said nucleic acid molecule comprises deoxyribonucleic acid (DNA) or Ribonucleic acid (RNA) which comprises monomers associated with said plurality of monomers, and wherein said method further comprises using said signals or changes thereof to identify said monomers thereby determining said sequence of said nucleic acid molecule or portion thereof.

120. The method of claim 114, wherein said electrical current is tunneling current.

121. The method of claim 114, wherein said individual monomers have labels that are different from one another.

122. The method of claim 114, wherein said one or more electrode pairs are used to detect said signals or changes thereof based at least in part on said labels while said sample molecule or portion thereof flows through said nano-gap.

123. The method of claim 114, wherein a flow or movement of said sample molecule is facilitated with an aid of said molecular motor.

124. The method of claim 123, wherein said molecular motor comprises an enzyme.

125. The method of claim 114, wherein said sequence of said sample molecule or portion thereof is determined at an accuracy of at least about 97%.

126. The method of claim 114, wherein said chip comprises an array of nano-gap sensors, wherein an individual nano-gap sensor of said array comprises a solid state membrane having at least one nano-gap which comprises electrodes that are adapted to generate said signals upon flow of electrical current thereacross.

127. The method of claim 126, wherein said solid state membrane is at least partially formed of a material selected from the group consisting of metallic materials and semiconductor materials.

128. The method of claim 126, wherein said solid state membrane has a thickness between about 10 nanometers (nm) and about 40 nm.

129. The method of claim 114, wherein said one or more electrode pairs are coupled to an electric circuit, and wherein said nano-gap sensor is coupled to an integrated circuit that processes said signals or changes thereof.

130. The method of claim 126, wherein said array of nano-gap sensors has a density greater than or equal to about 500 sensors per 1 mm².

131. The method of claim 114, wherein said nano-gap has an inter-electrode capacitance of less than about 0.1 picofarad (pF).

132. The method of claim 114, wherein said labels comprise tunneling labels or hopping labels.

133. The method of claim 114, further comprising determining epigenetically modified bases of said sample molecule using said signals or changes thereof.

134. The method of claim 114, wherein a flow or movement of said sample molecule is facilitated without an aid of a molecular motor.
